(19) United States
(12) Patent Application Publication
(10) Pub. No.: US 2007/0020269 A1
(43) Pub. Date: Jan. 25, 2007

(54) Phosphokinase and the usage thereof

(76) Inventors: Xiaoqing Sun, Shanghai (CN); Yingli Guo, Shanghai (CN)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/478,461

(22) Filed: Jun. 28, 2006

Related U.S. Application Data
(63) Continuation of application No. PCT/CN03/01164, filed on Dec. 31, 2003.

(51) Int. Cl. A61K 39/395; C07H 21/04; C12P 21/06; C12N 9/12

(52) U.S. Cl. 424/146.1; 435/69.1; 435/194; 435/320.1; 435/325; 530/388.26; 536/23.2

(57) ABSTRACT

The present invention relates to a novel phosphokinase, RX50 protein, polynucleotide encoding RX50 protein and the recombinant method of producing the same. RX50 protein can interact with p21 and cyclin D3, and inhibit the transcription of p53. Therefore, it can be used as a drug target for new drug selection.

This application is a continuation or International application No. PCT/CN2003/001164, filed Dec. 31, 2003, the content of which is herein incorporated in its entirety by reference.

### FIELD OF INVENTION

The present invention relates to biotechnological and medical field. More particular, the present invention relates to a novel phosphokinase, RX50 protein, and the polynucleotide encoding RX50. Further, it relates to the preparation and the usage of RX50 protein and the polynucleotide, as well as the composition containing the RX50 protein.

### TECHNICAL BACKGROUND

The high-quality drug target gene (or drug target) is the resource of new drug development. Although the completion of human genome project has shown a promising prospect of treatment of diseases, the genes themselves are not certainly drug targets except that some big protein moleculars can be used as drugs. Several indispensable elements in the linkage from gene to new drugs are still missing. The study of gene function is the key step to develop a gene into a drug target, because it can discover the secrets behind the human health and diseases to find out the most important genes that are disease-related.

Numerous overseas pharmaceutical companies have found that although it is possible to discover many potential drug target gene merely based on the gene sequence data and analysis of bio-informatics, these genes are classified as low-quality drug targets. Facing such huge number of low-quality drug targets, the pharmaceutical researchers found themselves helpless. A high-quality drug target, which is reliable for drug development can be screened out only after numerous studies of the gene function and gene verification. Therefore, the studies on functional genomics have huge values of applicability and business prospects.

Among the 5000 genes that are useful as targets; the phosphokinases are generally deemed as gene targets for drug screening because they have considerable sequence conservation.

Phosphokinase, phosphatase, protease and the various receptors are called as one class of targets. The phosphoester group on the ATP or GTP is transferred by phosphokinase onto the amino acid residues of the substrate protein, thereby catalyzing phosphorylation of protein. The phosphorylation and dephosphorylation of protein are one of the important means to regulate the function and/or activity of protein. For example, MAPK and some transcription factors, such as CREB, Jun are active in the phosphorylation state and inactive in the non-phosphorylation state. On the contrary, the transcription factor IκBα are inactive in the phosphorylation state and active in the non-phosphorylation state.

Up until now, a lot of phosphokinases have been discovered but few human phosphokinases are known. Due to the close relationship between phosphokinases and various physiological activities, such as cell division, there is a keen need in the art to develop new phosphokinases.

### SUMMARY OF INVENTION

One purpose of the invention is to provide novel phosphokinase-RX50 protein and the fragments, analogs and derivatives thereof.

The another purpose of the invention is to provide a polynucleotide encoding said proteins.

Still another. The last purpose of the invention is to provide a method for preparing said proteins and the usage of the proteins, and their encoding sequences.

In the 1st aspect, the invention provides the isolated RX50 protein, which comprise a polypeptide having the amino acid sequence of SEQ ID NO: 2, and the conservative variants, active fragments, and active derivatives thereof having activity of kinase.

Preferably, the protein is selected from the group consisting of:

(a) a polypeptide having the amino acid sequence of SEQ ID NO: 2;

(b) a polypeptide which is derived from polypeptide (a) by substitution, deletion or insertion of one or more (such as 1-10, and preferably 1-8) amino acid residues and which has the function of phosphorylation. More preferably, the polypeptide consists of the amino acid sequence as shown in SEQ ID NOs: 2.

In the 2nd aspect, the invention provides an isolated polynucleotide encoding the above RX50 protein.

Preferably, the polynucleotide encodes a protein comprising the amino acid sequence of SEQ ID NO: 2. More preferably, the polynucleotide comprises the nucleotide sequence of 1-1353 of SEQ ID NO: 1.

In the 3rd aspect, it provides a vector comprising the above polynucleotide encoding RX50, and a host cell transformed with the vector or directly transformed with the above polynucleotide.

In the 4th aspect, it provides a method for producing RX50 protein, which comprises:

(a) culturing the above transformed host cell under the expression conditions;

(b) isolating the protein having the amino acid sequence of SEQ ID NO: 2 from the culture.

In the 5th aspect, it provides an antibody specifically binding RX50 protein.

In the 6th aspect, it provides a pharmaceutical composition comprising a safe and efficient amount of RX50 protein and a pharmaceutically acceptable carrier.

### DESCRIPTION OF DRAWINGS

The following figures are used for illustration of the embodiments of the invention, not for the limitation of the scope of invention defined by the appended claims of the application.

FIG. 1 shows the result of RX50 sequence analysis.

FIG. 2 shows the homology comparison of RX50 and murine protein.

FIG. 3 shows the interaction between RX50 and endogenous p21 wherein lane A is control and lane B is RX50.

FIG. 4 shows the interaction between RX50 and cyclin D3.

The following substances are transfected into 293T cell: 1) flag-RX50; 2)flag-RX50 and HA-cyclin D3; 3)flag-RX50 and myc-p21; and 4)flag-RX50, myc-p21 and HA-cyclin D3. The binding between RX50 and cyclin D3 is relatively weak (*) while the binding between RX50 and cyclin D3 becomes very strong (**) when p21 is over-expressed.

FIG. 5 shows the phosphorylation activity of wild type and mutant RX50.

FIG. 6 shows the inhibition of p53 transcription by RX50.

FIG. 7 shows the inhibition of TNF-induced NF-KB transcription activity by RX50.

FIG. 8 shows the position of various p21 truncates.

FIG. 9 shows the site of interaction between RX50 and various p21 truncates.

### DETAILED DESCRIPTION

After comprehensive and extensive study, the inventors have first isolated and identified the full-length cDNA of a new human phosphokinase RX50, which encodes RX50 protein having 451 amino acid residues. RX50 protein has domains of phosphokinase and the experiment of self-phosphorylation has proven that RX50 protein is indeed a phosphokinase. On the basis of these studies, the inventors completed this invention.

The results of yeast dual hybridization experiments and co-immunoprecipitation experiments have proven that the direct interaction exists between RX50 and p21, as well between RX50 and Cyclin D3. The binding between RX50 and Cyclin D3 was enhanced by co-transformation of p21. Further, the different RX50 and p21 truncated variants were prepared and it was confirmed that RX50 interacts with 40-60 aa of p21. Moreover, RX50 inhibited the transcription activity of p50 as well as the TNF-induced NF-κB transcription activity.

As used herein, the term “phosphokinase RX50”, “RX50 protein”, or “RX50 polypeptide” are exchangeable, referring to a protein or polypeptide comprising or essentially consisting of the amino acid sequence of RX50 protein (SEQ ID NO: 2). The term includes RX50 protein with or without the starting Met residue, as well as the RX50 protein with or without signal peptide.

As used herein, the term “isolated” refers to a substance which has been isolated from the original environment. For naturally occurring substance, the original environment is the natural environment. E.g., the polynucleotide and polypeptide in a naturally occurring state in the viable cells are not isolated or purified. However, if the same polynucleotide and polypeptide have been isolated from other components naturally accompanying them, they are isolated or purified.

As used herein, the terms “isolated RX50 protein or polypeptide” mean that RX50 polypeptide does not essentially contain other proteins, lipids, carbohydrate or any other substances associated therewith in nature. The artisans can purify RX50 protein by standard protein purification techniques, especially FPLC.

The polypeptide of invention may be a recombinant, natural, or synthetic polypeptide, preferably a recombinant polypeptide. The polypeptide of invention may be a purified natural product or a chemically synthetic product. Alternatively, it may be produced from prokaryotic or eukaryotic hosts, such as bacteria, yeast, higher plant, insect, and mammalian cells, using recombinant techniques. According to the host used in the recombinant production, the polypeptide may be glycosylated or non-glycosylated. The polypeptide of invention may or may not comprise the starting Met residue.

The invention further comprises the fragments, derivatives and analogues of RX50 protein. As used in the invention, the terms “fragment”, “derivative” and “analogue” mean the polypeptide that essentially retains the same biological functions or activity of RX50 protein of the invention. The fragment, derivative or analogue of the polypeptide of invention may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues include a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as a leader or secretary sequence or a sequence which is employed for purification of the mature polypeptide or a proprotein sequence, e.g., a fusion protein formed with IgC fragment. Such fragments, derivatives and analogs are deemed to be within the scope of those skilled in the art from the teachings herein.

In the present invention, the term “RX50 protein” refers to a full-length polypeptide having the activity of RX50 protein comprising the amino acid sequence of SEQ ID NO: 2, or the mature polypeptide thereof. The term also comprises the variants of said amino acid sequence which have the same function of RX50 protein. These variants include, but are not limited to, deletions, insertions and/or substitutions of one or more amino acids (typically 1-50, preferably 1-30, more preferably 1-20, most preferably 1-10), and addition of one or more amino acids (typically less than 20, preferably less than 10, more preferably less than 5) at C-terminal and/or N-terminal. For example, the protein functions are usually unchanged when an amino residue is substituted by a similar or analogous one. Further, the addition of one or several amino acids at C-terminal and/or N-terminal will not change the function of protein. The term also includes the active fragments and derivatives of RX50 protein.

The variants of polypeptide include homologous sequences, allelic variants, natural mutants, induced mutants, proteins encoded by DNA which hybridizes to RX50 DNA under high or low stringency conditions as well as the polypeptides or proteins retrieved by antisera raised against RX50 protein. The present invention also provides other polypeptides, e.g., fusion proteins, which include the RX50 polypeptide or fragments thereof. In addition to substantially full-length polypeptide, the soluble fragments of RX50 polypeptide are also included. Generally, these fragments comprise at least 10, typically at least 30, preferably at least 50, more preferably at least 80, most preferably at least 100 consecutive amino acids of RX50 polypeptide.

The present invention also provides the analogues of RX50 protein. Analogues can differ from naturally occurring RX50 protein by amino acid sequence differences or by modifications that do not affect the sequence, or by both. These polypeptides include genetic variants, both natural and induced. Induced variants can be made by various techniques, e.g., by random mutagenesis using irradiation or exposure to mutagens, or by site-directed mutagenesis or other known molecular biologic techniques. Also included are analogues which include residues other than those naturally occurring L-amino acids (e.g., D-amino acids) or non-naturally occurring or synthetic amino acids (e.g., beta- or gamma-amino acids). It is understood that the polypeptides of the invention are not limited to the representative polypeptides listed hereinabove.

Modifications (which do not normally alter primary sequence) include in vivo or in vitro chemical derivation of polypeptides, e.g., acelylation, or carboxylation. Also included are modifications of glycosylation, e.g., those made by modifying the glycosylation patterns of a polypeptide during its synthesis and processing or in the further processing steps, e.g., by exposing the polypeptide to enzymes which affect glycosylation (e.g., mammalian glycosylating or deglycosylating enzymes). Also included are sequences that have phosphorylated amino acid residues, e.g., phosphotyrosine, phosphoserine, phosphothronine, as well as sequences that have been modified to improve their resistance to proteolytic degradation or to optimize solubility properties.

In the invention, “RX50 conservative mutant” means a polypeptide formed by substituting at most 10, preferably at most 8, more preferably 5, and most preferably at most 3 amino acids with the amino acids having substantially the same or similar property, as compared with the amino acid sequence of SEQ ID NO: 2. Preferably, these conservative mutants are formed by the substitution according to Table 1.

TABLE 1

| Initial | | Preferred |
|---|---|---|
| residue | Representative substitution | substitution |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

The polynucleotide according to the invention may be in the forms of DNA and RNA. DNA includes cDNA, genomic DNA, and synthetic DNA, etc., in single strand or double strand form. A single strand DNA may be an encoding strand or non-encoding strand. The coding sequence for mature polypeptide may be identical to the coding sequence shown in SEQ ID NO: 1, or may be a degenerate sequence. As used herein, the term “degenerate sequence” means an sequence which encodes a protein or peptide comprising a sequence of SEQ ID NO: 2 and which has a nucleotide sequence different from the sequence of coding region in SEQ ID NO: 1.

The sequences encoding the mature RX50 protein (SEQ ID NO: 2) include those encoding only the mature polypeptide, those encoding mature polypeptide plus various additional encoding sequence, the encoding sequence for mature polypeptide plus the non-encoding sequence and optional additional encoding sequence.

The term “polynucleotide encoding the protein” includes the polynucleotide encoding said protein and the polynucleotide comprising additional and/or non-encoding sequence.

The invention further relates to the variants of the hereinabove polynucleotides which encode a polypeptide having the same amino acid sequence of invention, or its fragment, analogue and derivative. The variant of the polynucleotide may be a naturally occurring allelic variant of the polynucleotide or a non-naturally occurring variant of the polynucleotide. Such nucleotide variants include substitution, deletion, and insertion variants. As known in the art, the allelic variant is a substitution form of polynucleotide, which may be a substitution, deletion, and insertion of one or more nucleotides without substantially changing the functions of the encoded polypeptide.

The present invention further relates to polynucleotides, which hybridize to the hereinabove-described sequences, if there is at least 60%, preferably at least 70%, more preferably at least 80%, and most preferably at least 90% identity between the sequences. The present invention particularly relates to polynucleotides, which hybridize under stringent conditions to the polynucleotides of the invention. As herein used, the term “stringent conditions” means the following conditions: (1) hybridization and washing under low ionic strength and high temperature, such as 0.2×SSC, 0.1% SDS, 60° C.; (2) hybridization after adding denaturants, such as 50% (v/v) formamide, 0.1% bovine serum/0.1% Ficoll, 42° C.; or (3) hybridization of two sequences sharing at least 90%, preferably 95% homology. Further, the polynucleotides which hybridize to the hereinabove described polynucleotides encode a polypeptide which retains the same biological function or activity as the mature protein shown in SEQ ID NO: 2.

The invention also relates to nucleic acid fragments hybridized with the hereinabove sequence. As used in the present invention, the length of the “nucleic acid fragment” is at least 15 bp, preferably at least 30 bp, more preferably at least 50 bp, and most preferably at least 100 bp. The nucleic acid fragment can be used in the amplification techniques of nucleic acid, e.g., PCR, so as to determine and/or isolate the polynucleotide encoding RX50 protein.

These polypeptide and polynucleotide of the invention is preferably provided in an isolated form or more preferably is purified to be homogenous.

The full-length RX50 nucleotide sequence or its fragment can be prepared by PCR amplification, recombinant method and synthetic method. For PCR amplification, one can obtain said sequences by designing primers based on the nucleotide sequence disclosed herein, especially the ORF, and using cDNA library commercially available or prepared by routine techniques in the art as a template. When the sequence is long, it is usually necessary to perform two or more PCR amplifications and link the amplified fragments together correctly.

Once the sequence is obtained, one can produce lots of the sequences by recombinant methods. Usually, said sequence is cloned into a vector which is then transformed into a host cell. The sequence is isolated from the amplified host cells using conventional techniques.

Further, the sequence can be synthesized, especially when the fragment is short. Typically, several small fragments are synthesized and linked together to obtain a long sequence.

It is completely feasible to chemically synthesize the DNA sequence encoding the protein of invention, or the fragments or derivatives thereof. Then, the DNA sequence can be introduced into the various DNA molecules (such as vectors) and cells available in the art. In addition, the mutation can be introduced into the protein sequence by chemical synthesis.

The method of amplification of DNA/RNA by PCR is preferably used to obtain the gene of the invention. The primers used in PCR can be properly selected according to the polynucleotide sequence information of invention disclosed herein and synthesized by the conventional methods. The amplified DNA/RNA fragments can be isolated and purified by conventional methods such as gel electrophoresis.

The invention further relates to a vector comprising the polynucleotide of the invention, a genetic engineered host cell transformed with the vector of the invention or directly with the sequence encoding RX50 protein, and the method for producing the protein of invention by recombinant techniques.

The recombinant human RX50 protein can be expressed or produced by the conventional recombinant DNA technology (Science, 1984; 224: 1431), using the polynucleotide sequence of invention. Generally, it comprises the following steps:

(1) transfecting or transforming the appropriate host cells with the polynucleotide or its variants encoding RX50 protein of the invention or the vector containing said polynucleotide;

(2) culturing the host cells in an appropriate medium;

(3) isolating or purifying the protein from the medium or cells.

In the present invention, the polynucleotide sequences encoding RX50 protein may be inserted into a recombinant expression vector. The term “expression vector” refers to a bacterial plasmid, bacteriophage, yeast plasmid, plant virus or mammalian cell virus, such as adenovirus, retrovirus or any other vehicle known in the art. Vectors suitable for use in the present invention include, but are not limited to, the T7-based expression vector for expression in bacteria, the pMSXND expression vector for expression in mammalian cells and baculovirus-derived vectors for expression in insect cells. On the whole, any plasmid or vector can be used to construct the recombinant expression vector as long as it can replicate and is stable in the host. One important feature of expression vector is that the expression vector typically contains an origin of replication, a promoter, a marker gene as well as the translation regulatory components.

The methods known by the artisans in the art can be used to construct an expression vector containing the DNA sequence of RX50 and appropriate transcription/translation regulatory components. These methods include in vitro recombinant DNA technique, DNA synthesis technique, in vivo recombinant technique and so on. The DNA sequence is efficiently linked to the proper promoter in an expression vector to direct the synthesis of mRNA. The exemplary promoters are lac or trp promoter of E. coli; P_{L} promoter of lamda-phage; eukaryotic promoter including CMV immediate early promoter, HSV thymidine kinase promoter, early and late SV40 promoter, LTRs of retrovirus and some other known promoters which control the gene expression in the prokaryotic cells, eukaryotic cells or virus. The expression vector may further comprise a ribosome-binding site for initiating the translation, transcription terminator and the like.

The expression vector preferably comprises one or more selective marker genes to provide a phenotype for selecting the transformed host cells, e.g., the dehydrofolate reductase, neomycin resistance gene and GFP (green flurencent protein) for eukaryotic cells, as well as tetracycline or ampicillin resistance gene for E. coli.

The vector containing said DNA sequence and proper promoter or regulatory elements can be transformed into appropriate host cells to express the protein.

The “host cell” includes prokaryote, e.g., bacteria; primary eukaryote, e.g., yeast; advanced eukaryotic, e.g., mammalian cells. The representative examples are bacterial cells, e.g., E. coli, Streptomyces, Salmonella typhimurium; fungal cells, e.g., yeast; plant cells; insect cells e.g., Drosophila S2 or Sf9; animal cells e.g., CHO, COS, or 293 cells, etc.

Transcription of the polynucleotide in higher eukaryotes is increased by inserting an enhancer sequence into the vector. Enhancers are cis-acting elements of DNA, usually about 10-300 bps, that act on a promoter to increase gene transcription. Examples include SV40 enhancer on the late side of replication origin 100 to 270 bp, the polyoma enhancer on the late side of replication origin, and adenovirus enhancers.

The artisans know clearly how to select appropriate vectors, promoters, enhancers and host cells.

Recombinant transformation of host cell with the DNA might be carried out by conventional techniques known to the artisans. Where the host is prokaryotic, e.g., E. coli, the competent cells capable of DNA uptake, can be prepared from cells harvested after exponential growth phase and subsequently treated by the CaCl₂ method using known procedures. Alternatively, MgCl₂ can be used. The transformation can also be carried out by electroporation. When the host is an eukaryote, transfection of DNA such as calcium phosphate co-precipitates, conventional mechanical procedures e.g., micro-injection, electroporation, or liposome-mediated transfection may be used.

The transformants are cultured conventionally to express RX50 protein of invention. According to the used host cells, the medium for cultivation can be selected from various conventional mediums. The host cells are cultured under a condition suitable for its growth until the host cells grow to an appropriate cell density. Then, the selected promoter is induced by appropriate means (e.g., temperature shift or chemical induction) and cells are cultured for an additional period.

In the above methods, the recombinant polypeptide may be included in the cells, or expressed on the cell membrane, or secreted out. If desired, the physical, chemical and other properties can be utilized in various isolation methods to isolate and purify the recombinant protein. These methods are well-known to the artisans and include, but are not limited to conventional renaturation treatment, treatment by protein precipitant (e.g., salt precipitation), centrifugation, cell lysis by osmosis, sonication, supercentrifugation, molecular sieve chromatography or gel chromatography, adsorption chromatography, ion exchange chromatography, HPLC, and any other liquid chromatography, and the combination thereof.

The recombinant human RX50 protein or polypeptide have various uses including, but not to be limited to: screening out antibodies, polypeptides or ligands as agonists or antagonists of RX50. The expressed recombinant RX50 protein can be used to screen polypeptide library to find out therapeutically valuable polypeptide molecules which inhibit or activate RX50 protein.

In another aspect, the invention also includes polyclonal and monoclonal antibodies (mAbs), preferably mAbs, which are specific for polypeptides encoded by RX50 DNA or fragments thereof. By “specificity”, it means an antibody which binds to the RX50 gene products or a fragments thereof. Preferably, the antibody binds to the RX50 gene products or fragments thereof and does not substantially recognize nor bind to other antigenically unrelated molecules. Antibodies which bind to RX50 and block RX50 protein and those which do not affect the RX50 function are included in the invention. Antibodies which bind to modified or unmodified RX50 protein are also included in the invention.

The present invention includes not only intact monoclonal or polyclonal antibodies, but also immunologically-active antibody fragments, e.g., a Fab′ or (Fab)₂ fragment, an antibody light chain, an antibody heavy chain, a genetically engineered single chain Fv molecule, or a chimerical antibody, e.g., an antibody which contains the binding specificity of a murine antibody, but the remaining portion of which is of human origin.

The antibodies in the present invention can be prepared by various techniques known in the art. E.g.,, purified RX50 gene products, or its antigenic fragments can be administrated to animals (e.g., rabbit, mice and rat) to produce polyclonal antibodies. Similarly, cells expressing RX50 or its antigenic fragments can be used to immunize animals to produce antibodies. The antibody of the invention can be monoclonal antibodies (mAbs). The mAbs can be prepared using hybridoma technique. Antibodies comprise those which block RX50 function and those which do not affect RX50 function. Antibodies can be produced by routine immunology techniques and using fragments or functional regions of RX50 gene product prepared by recombinant methods or synthesized by a polypeptide synthesizer. The antibodies binding to unmodified RX50 gene product can be produced by immunizing animals with gene products produced by prokaryotic cells (e.g., E. coli), and the antibodies binding to post-translationally modified forms thereof can be acquired by immunizing animals with gene products produced by eukaryotic cells (e.g., yeast or insect cells).

The substances which act with RX50 protein, e.g., receptors, inhibitors, agonists and antagonists, can be screened out by various conventional techniques, using RX50 protein. Usually, the screen models suitable for high-flux screening on molecular or cellular level are established and the related studies on the high-flux screening are carried out. Using E. coli or Bacula virus expression system, the active fragments of tyrosine phosphoesterase are cloned and expressed. The recombinant proteins are isolated and purified. By using these recombinant enzymes, the screening model on the molecular level is established. Various crude extracts and pure compounds from traditional herbal materials can be screened out so as to find out efficient active portions or pure compounds. The activity can be used to direct the isolation of monomer from efficient portions. The small-molecule inhibitors which are obtained by the high flux screening method can be used to detect the effects on RX50 to determine the specificity of inhibition by the small-molecule inhibitors. The small-molecule inhibitors which are obtained by the high flux screening method can be used to detect the inhibition effects on the cell level.

The RX50 protein, and its antibody, inhibitor, agonist or antagonist of the invention provide different effects when administrated in therapy. Usually, these substances are formulated with a non-toxic, inert and pharmaceutically acceptable aqueous carrier. The pH typically is about 5-8, preferably 6-8, although pH may alter according to the property of the formulated substances and the diseases to be treated. The formulated pharmaceutical composition is administrated in conventional routes including, but not limited to, oral, intramuscular, intraperitoneal, intravenous, subcutaneous, intradermal or topical administration.

The invention also provides a pharmaceutical composition comprising safe and effective amount of RX50 protein in combination with a pharmaceutically acceptable carrier. Such a carrier includes but is not limited to saline, buffer solution, glucose, water, glycerin, ethanol, or the combination thereof. The pharmaceutical formulation should be suitable for delivery method. The pharmaceutical composition may be in the form of injections that are made by conventional methods, using physiological saline or other aqueous solution containing glucose or auxiliary substances. The pharmaceutical compositions in the form of tablet or capsule may be prepared by routine methods. The pharmaceutical compositions, e.g., injections, solutions, tablets, and capsules, should be manufactured under sterile conditions. The active ingredient is administrated in therapeutically effective amount, e.g., about 1 ug-10 mg/kg body weight per day. Moreover, the protein of the invention can be administrated together with other therapeutic agent.

When the RX50 protein of the invention are used as a pharmaceutical, the therapeutically effective amount of the polypeptides are administrated to mammals. Typically, the therapeutically effective amount is at least about 1 ug/kg body weight and less than about 10 mg/kg body weight in most cases, and preferably about 10 ug-0.5 mg/kg body weight. Of course, the precise amount will depend upon various factors, such as delivery methods, the subject health, and the like, and is within the judgment of the skilled clinician.

A method of detecting RX50 protein in a sample by utilizing the antibody specifically against RX50 protein comprises the steps of: contacting the sample with the antibody specifically against RX50 protein; observing the formation of antibody complex which indicates the presence of RX50 protein in the sample.

The main advantages of the invention are as follows. The RX50 protein of the invention is a novel phosphokinase that interacts with p21 and cyclin D3. Therefore, it can be used as a drug target to screen out small-molecule compounds, to establish models for screening out drugs targeting RX50, and to find out small-molecule compounds that regulate the kinase activity of RX50 protein, thereby improving the efficiency and specificity of drug screen and providing a new way for the diagnosis and treatment of diseases such as cancer.

The invention is further illustrated by the following examples. It is appreciated that these examples are only intended to illustrate the invention, but not to limit the scope of the invention. For the experimental methods in the following examples, they are performed under routine conditions, e.g., those described by Sambrook. et al., in Molecule Clone: A Laboratory Manual, New York: Cold Spring Harbor Laboratory Press, 1989, or as instructed by the manufacturers, unless otherwise specified.

### EXAMPLE 1

### Screening for the Gene RX50

PFAM/Profile of the bioinformatics was used to preferably screen phosphokinase, phosphatase, proteinase and monotransmembrane receptor from cDNA library of the non-annotated or partially annotated “new” genes which were established on the basis of the ESTs in Genebank. By this means, a new gene containing a phosphokinase domain without any annotation, was predicted and designated as RX50.

### EXAMPLE 2

### Amplification of the Gene RX50

In order to obtain the full-length RX50 gene, conventional PCR was performed using the mixed RNAs extracted from human tissues by conventional methods as template and the following primers:

Forward primer: 5′ ggccaatccg gccatgcacg gttactttgg ctgcaatgc 3′ (SEQ ID NO:3)

Reverse primer: 5′ ggcctctaag gcctcagtgc ttgctgtttg atagactttt gcc 3′ (SEQ ID NO:4).

The above primers contained SfiI shuffling cloning site, a start codon and a stop codon. The coding sequence of RX50 was between the restriction enzyme sites.

A band of about 1.3 kb was obtained by PCR amplification using this pair of primers. The fragment of this size was purified, cloned into a vector, and sequenced, thereby obtaining the full-length sequence of the gene RX50 which was 1356 bp (SEQ ID NO: 1).

### EXAMPLE 3

### Sequence Analysis and Location of RX50

The sequence of 1356 bp cloned in example 1 comprised the entire coding region (1-1353) of RX50, which encoded a RX50 protein (SEQ ID NO: 2) consisting of 451 amino acids. Amino acid residues 123-146, i.e.LGEGSYATVYKGKSKVNGKLVALK of SEQ ID NO:2 constituted a ATP binding site, and amino acid residues 117-401 constituted a kinase domain (FIG. 1).

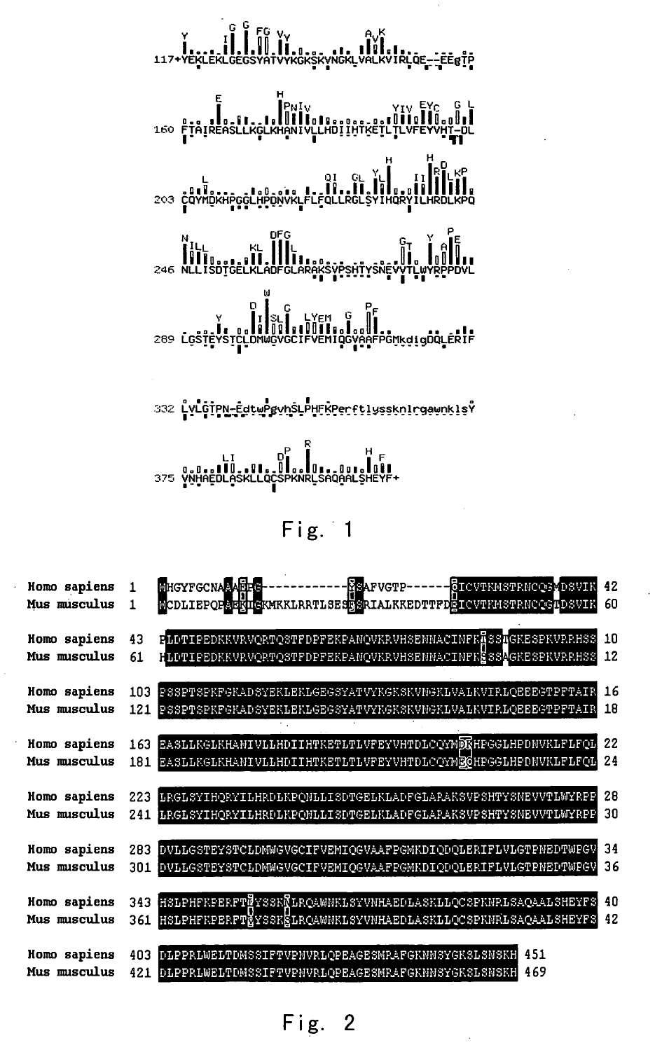

According to the information of its ESTs, RX50 gene was mapped onto human chromosome 7q21.13. The human RX50 was subjected to homology alignment and it was found that RX50 shared 91% homology with a mice protein whose function was unknown (FIG. 2).

### EXAMPLE 4

### Screening for the Protein Interacting with RX50

In this example, Ga14 yeast two-hybrid system (Clontech Corporation) was used to screen for the protein that interacted with RX50. The process was as follows. The RX50 gene verified by sequencing was transferred into a modified fusion plasmid pGBKT 7 using the SfiI shuffling cloning site. This recombinant plasmid was used as a bait to make a large scale screen of Hela, lymph and fetal brain libraries by transformation and mating, respectively. P21 and cyclin D3 positive clones were obtained by both methods. That is to say, two known proteins p21 and cyclin D3 which interacted with RX50 were screened out by the yeast two-hybrid method.

There was a conservative domain of Cdc2-related protein kinase in the region of amino acid position 117-401 of the putative protein sequence deduced from the nucleotide sequence of RX50. The protein cyclin D3 was screened out by the yeast two-hybrid method described above, and it was known that cyclin binded to specific CDK to regulate cell cycle. Therefore, the information indicated that RX50 was a new member of CDK family, which was related to the regulation of p21.

In order to further identify the specific cyclin interacting with RX50, it was verified in the yeast two-hybrid system whether RX50 interacted with the other cyclins, including cyclin A, cyclin B, cyclin C, cyclin D1, cyclin D2, cyclin E1, cyclin E2, cyclin F, cyclin G, cyclin H, cyclin I and cyclin K. The results showed that RX50 only interacted with cyclin D3. So cyclin D3 was a specific cyclin for RX50.

### EXAMPLE 5

### Expression of RX50 and the Interaction of RX50 with p21 and Cyclin D3 in Mammals Measured by Co-immunoprecipitation

Since the yeast two-hybrid system itself might give a false positive result, co-immunoprecipitation was employed to determine the relationship between RX50 and p21 and between RX50 and cyclin D3 in the mammalian animals. The process was as follows: A SfiI site was added to the multiple cloning site of pcDNA3.1 (Invitrogen Corporation) by using conventional methods. Next, flag-tag, myc-tag and HA-tag were introduced into the N-terminus of the SfiI site, respectively, to produce pcDNA3.1 vectors each of which carried one of the tags described above. RX50 obtained by PCR amplification (example 2), genes p21 and cyclin D3 were cloned respectively into the pcDNA3.1 eukaryotic expression vectors carrying flag-tag, myc-tag and HA-tag by using the SfiI site. The protein expression of RX50, p21 and cyclin D3 were measured by Western-blot using monoclonal antibody of anti-flag, anti-myc and anti-HA (Sigma Corporation).

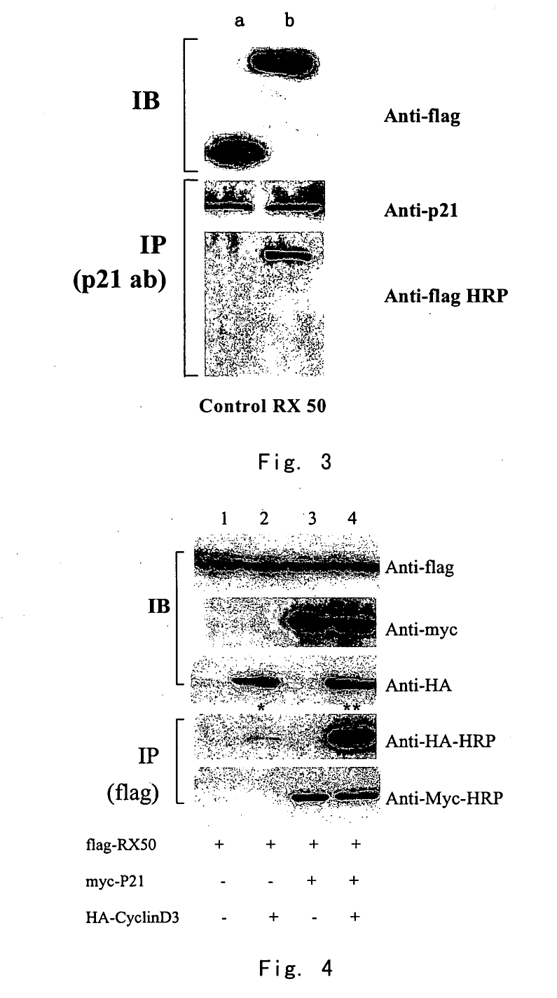

The results were shown in FIG. 3 and FIG. 4. The results showed that Flag-RX50, myc- p21 and HA-cyclin D3 were expressed well at a stable level.

First, the cell line 293T was transfected with RX50, lysed by addition of lysate after 24 hrs of culture. The supernatant was collected by centrifugation, and then immunoprecipitated with anti-p21 antibody. The product of co-immunoprecipitation was electrophoresised on SDS-PAGE, transferred onto a nitro cellulose membrane, and then subjected to immunoblot with a enzyme-labeled anti-flag antibody. A specific hybrid band of 50 kDa was observed, in consistence with the protein encoded by RX50. This result indicated that this band was corresponding to the protein encoded by RX50. Therefore, RX50 interactrf with the endogenous p21.

To clarify the relationship of RX50, p21 and cyclin D3, RX50 and cyclin D3 were used to co-transfect 293T cells. The transfected cells were allowed to grow 24 hrs before lysis. The supernatant was collected by centrifugation. The cell pellets were subjected to immunoprecipitation with anti-flag antibody and Protein G. The product of co-immunoprecipitation was electrophoresised on SDS-PAGE, transferred onto a nitro cellulose membrane, and then subjected to immunoblot with a enzyme-labeled anti-HA antibody. It was observed that the binding between RX50 and cyclin D3 was weak (FIG. 4, loan 2).When RX50, cyclin D3 and p21 were used to co-transfect the cells, it was observed that when p21 was overexpressed, the binding of RX50 and cyclin D3 was dramatically enhanced. This result indicated that cyclin D3 interacted with p21 to form a trimer. It also demonstrated that RX50 interacted with p21 (lane 3).

### EXAMPLE 6

### Truncated Mutants of p21

In order to obtain differently truncated p21, PCR amplification was performed using primers corresponding to amino acid residue 20, 40, 60, 91, 89 and a primer corresponding to the C-terminus. The products of the amplification were cloned into the pcDNA3 eukaryotic expression vector with myc-tag, respectively, to produce the truncated mutants of p21, including p21-D2, p21-D3, p21-C and p21-N (see, FIG. 8).

### EXAMPLE 7

### The Interacting Regions of RX50 and p21 in Mammals Determined By Co-immunoprecipitation

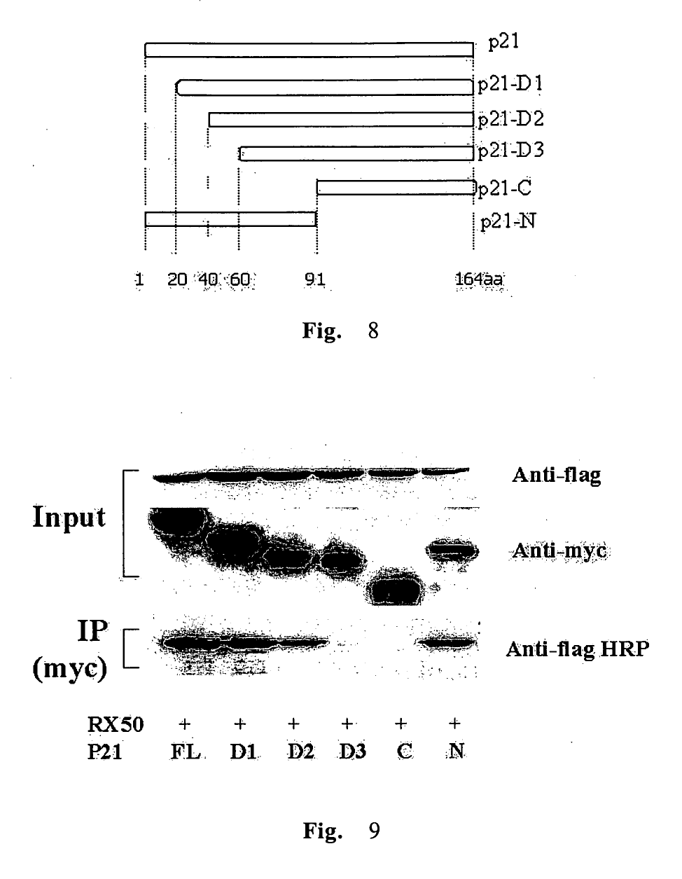

In order to determine the interacting regions of RX50 and p21, the 293T cells were co-transfected with RX50 and p21, RX50 and p21-D1, RX50 and p21-D2, RX50 and p21-D3, RX50 and p21-C, RX50 and p21-N, respectively. The transfected cells were allowed to grow 24 hrs before lysis. The supernatant was collected by centrifugation. The cell pellets were subjected to immunoprecipitation with anti-myc antibody and Protein G. The product of co-immunoprecipitation was electrophoresised on SDS-PAGE, transferred onto a nitro cellulose membrane, and then subjected to immunoblot with a enzyme-labeled anti-flag antibody. The result indicated that RX50 interacted with p21, p21-D1, p21-D2 and p21-N (FIG. 9, lanes 1, 2, 3, 6), whereas RX50 did not interact with p21-D3 and p21-C (FIG. 9, lanes 4, 5). It was inferred that the region of p21 interacting with RX50 was located between amino acid residue 40 to 60 of p21.

In addition, a serial of truncated mutants of RX50 were constructed by a similar method. The results of the co-immunoprecipitation showed that the region of RX50 interacting with p21 was located between amino acid residue 115-230 of RX50.

### EXAMPLE 8

### Phosphorylation of RX50

In this example, RX50 was verified to have the phosphorylation function of kinase by an in vitro self-phosphorylation experiment.

First, conventional specific-site mutagenesis was used to obtain the RX50 mutant RX50mut, which has a K→A mutation at amino acid residue 146 in the ATP binding site. The mutation was achieved by replacement of A, A at amino acid residues 436, 437 of SEQ ID NO:1 with G and C, respectively. Both wild-type and mutant RX50 were introduced into pcDNA3 eukaryotic expression vector carrying Flag-tag. The obtained recombinant vector was used to transfect 293T cells. The transfected cells were allowed to grow 24 hrs before lysis. The supernatant was collected by centrifugation. The cell pellets were subjected to immunoprecipitation with anti-flag antibody and Protein G. Half of the product of co-immunoprecipitation was subjected to Western-blot to identify the expression of RX50. Another half of the product was equilibrated with a kinase reaction buffer (20 mM Tris/HCL pH=7.4, 150 mM NaCl, 10 mM MnCl₂, 50 μM ATP, 10 mM MgCl₂), then 10 μCi γ-³²P ATP was added. The reaction was incubated at 30° C. for 30 min. Equal volume of 2×SDS-PAGE loading buffer was added to denature the sample at 95° C. for 5 min. The sample was collected by centrifugation and electrophoresised on 15% gradient SDS-PAGE gel. After electrophoresis, the gel was dried, and autoradiographed by X ray.

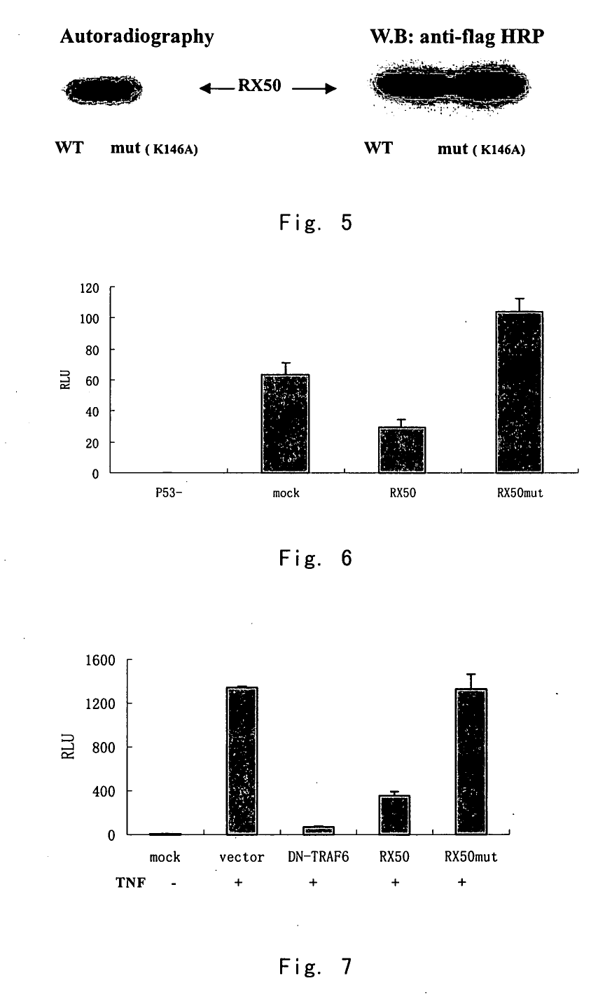

The result showed that RX50 could phosphorylate itself and had the activity of kinase. When the ATP binding site was mutated, the activity of the kinase was eliminated (FIG. 5).

### EXAMPLE 9

### The Effect of RX50 Overexpression on Some Reporter Genes

RX50 and mutant RX50 were employed to transfect different mammal cells, including the commonly used 293T cells, Jurkat cells, Saos cells and U20S cells. Several reporter genes associated with cancer and inflammation were also introduced into these cells. These reporter genes included:

A. p53-luciferase reporter gene

B. NFAT-luciferase reporter gene

C. NF-kB-luciferase reporter gene

D. AP1-luciferase reporter gene

The luciferase activity was measured after 24 hrs of transfection. Luciferase assay was very sensitive and thus produced an experiment error. Therefore, each experiment was repeated more than 3 times, and the data represented the average of more than 3 independent experiments.

The result showed that RX50 inhibited the transcriptional activity of p53 by over 50% in Saos cells, whereas this effect was not observed in the RX50 mutant (K146A) with a mutation at the ATP binding site (FIG. 6). Likewise, the result from the 293T cells showed that RX50 inhibited the transcriptional activity of NF-KB by over 60%, whereas the RX50 mutant (K146A) with a mutation at the ATP binding site did not have the inhibitory activity (FIG. 7).

This indicates that RX50 may be involved in the development and the regulatory mechanism of cancer and inflammation.

### EXAMPLE 10

### Preparation of the Rabbit Anti-RX50 Antibody

1 mg of RX50 protein (Example 5) was emulsified by grounding in Freund's complete adjuvant, and then injected at multiple sites on the beck of male New Zealand rabbit (body weight: about 2 kg). After 15 days, 1 mg of RX50 protein was emulsified by grounding in Freund's incomplete adjuvant, and injected again at multiple sites on the beck of the rabbit. After 30 days, the rabbit was boostered as described above with 1.5 mg of RX50 protein in Freund's incomplete adjuvant. After 15 days, the rabbit was boostered again with 1 mg of RX50 protein in Freund's incomplete adjuvant. After 15 days, blood was drawn from the carotid artery, placed without stirring at 4° C. overnight. Then the blood sample was centrifugated at 2,000 rpm for 3 min. The serum in the upper layer was the rabbit anti-RX50 antibody.

The result of the hybridization showed that the anti-RX50 antibody could specifically bind with the RX50 protein.

### DISCUSSION

P21 is a key negative regulatory factor for cell proliferation. P21 is a single-copy gene located on the short arm of chromosome 6 (6P21.2). The length of DNA is 85 kb having three exons whose length were 68, 450, and 1600 bp, respectively. The unique sequence for binding P53 were located in the promoter region of p21. Therefore, P21 and P53 were close related.

The gene mutation of P53 gene is most observed in the human malignant tumors. P53 locates on the band 4, region 1 of the short arm of Chromosome 17. P53 has two forms. Wild type P53 is an antioncogene. Under the normal conditions, DNA is damaged by the mutagenic agents, thereby quickly inducing the wild type p53 and activating p21 transcription. The cell cycle is blocked in G1 period and the cyclin (or PCNA) is bound to inhibit the DNA replication so that the damaged DNA can be repaired before the replication. The mutant p53 lacks the capacity to block the cell cycle after the DNA is damaged and has the activity to facilitate the malignant conversion. The tumor cells having no wild type p53 were resistant to apotosis so that the living of tumor cells is maintained and the resistance to chemotherapy and radiotherapy is increased. The ratio of p53 gene mutation is above 50% and P53 is often inactive in most human cancers such as Leukemia, lymphoma, sarcoma, cerebroma, mammary cancer, Gastrointestinal tract cancer and lung cancer.

P21 gene, as a downstream mediator of P53, performs some of the functions of P53 gene. P21 (Waf/Cip/Sid) protein directly binds CDK or cyclin-CDK complex, inhibits activity of various CDKs (CDK2, 4, and 6), and makes the cell cycle stop so that the cell has opportunity to repair the damaged DNA or correct the mistakes occurred in the DNA replication. In the several tumor tissue samples, the low expression and the deletion of p21 (Waf/Cip/Sid) protein is quite frequent. Further, because of the polymorphism of P21 gene, p21(Waf/Cip/Sid) also can independently involve many activity in the cell in the p53-independent routes, such as participating the differentiation of stem cell as well as the interaction with various cell transcription factors such as E2F, C/EBP-a, protein kinase Pim, calmodulin, GADD45 and so on.

Because of the close relationship between p21 and p53, the effect on p53 gene function by RX50 was further studied by using reporter gene system. The results showed that wild type RX50 could significantly reduce the activity of p53 in Soas cells. Because part of the p53 gene functions are achieved by the activation of p21 transcription, it implies that it is the binding between RX50 and p21 that inhibits part of p53 activities, suggesting RX50 participates the regulation of p21 and p53, which are two key negative regulatory factors of the cell cycle. Therefore, RX50 may have important values in the occurrence, development, diagnosis and treatment of various diseases such as tumor.

In the invention, the direct interaction between RX50 and Cyclin D3 was confirmed, and the existence of p21 could significantly enhance the binding between RX50 and Cyclin D3. Cyclins are the key proteins in the cell cycle. The most important task of cell cycle (including G1, S, G2 and M phases) is to completely replicate the genomic DNA into two copies in the DNA synthesis period (S phase) and to distribute two copies to two daughter cells in the dividing phase (M phase). Cyclins are in charge of the normal progress of cell cycle, while the regulation of cyclins is co-influenced by the cyclin dependent kinases or CDKs, and the opposing proteins such as p21, p16 and the like. If the CDKs are the accelerator of cell cycle, then p21, p16 and the like are the brake of cell cycle. The complex formed between Cyclin and CDKs activates the kinase activity of CDKs and makes the particular proteins phosphorylated, thereby further influencing the downstream proteins and participating the regulation of the switch of G1-S, G2-M in the cell cycle. However, when DNA is impaired or mistakes occur in the DNA replication, the cell cycle is timely stopped by p21, p16 and so on by blocking the cell cycle in G1 period. After the cell is repaired, the progress of cell cycle is recovered. If the two key checkpoints of G1-S and G2-M are out of control, the cell whose proliferation should be stopped or which should ongo phsiological apoptosis may enter into cell cycle , thereby initiating the malignant proliferation of tissue cells and causing various disorders, among which the development of tumor is most important.

Based on the deduced amino acid sequence from nucleotide sequence of RX50 gene, there is a Cdc2 related protein kinase conservative domain on the amino acid fragment of position 117-401. We have confirmed the direct interaction between RX50 and Cyclin D3 and the existence of p21 can significantly enhance the binding between RX50 and Cyclin D3, which are exciting results. From the research results recognized in the world, cyclin only binds to specific CDK to form a complex which activates the kinase activity of CDK so that it can regulate the cell cycle. Each cyclin only binds to the specific CDK. Therefore, it suggests that it is of great possibility that RX50 is an undiscovered member in the CDK family that is related to mechanism of p21 regulation. Because CDK is possibly the core of cell cycle regulatory device, the new kinase RX50 is possibly related with the cell cycle, proliferation, apoptosis and the development of tumor.

Once clarifying the mechanism of RX50 functions, one can use RX50 as a drug target to screen out small-molecule compounds, to establish models for screening out drugs targeting RX50, and to find out small-molecule compounds that regulate the kinase activity of RX50 protein, thereby improving the efficiency and specificity of drug screen and providing a new way for the diagnosis and treatment of diseases such as cancer.

All the documents cited herein are incorporated into the invention as reference, as if each of them is individually incorporated. Further, it would be appreciated that, in the above teaching of the invention, the skilled in the art could make certain changes or modifications to the invention, and these equivalents would still be within the scope of the invention defined by the appended claims of the present application.

What is claimed is:

1. An isolated protein comprising a polypeptide having the amino acid sequence of SEQ ID NOs: 2, the conservative variants, active fragments, and active derivatives thereof.

2. The protein of claim 1 wherein the protein is selected from the group consisting of:
(a) a polypeptide having the amino acid sequence of SEQ ID NO: 2;
(b) a polypeptide which is derived from polypeptide (a) by substitution, deletion or insertion of 1-10 amino acid residues and which has the function of phosphorylation.

3. An isolated polynucleotide encoding the protein as defined in claim 1.

4. The polynucleotide of claim 3 which encodes a protein having the amino acid sequence of SEQ ID NO: 2.

5. The polynucleotide of claim 3 which comprises the nucleotide sequence of 1-1353 of SEQ ID NO: 1.

6. A vector containing the polynucleotide of claim 3.

7. A genetically engineered host cell comprising the vector of claim 6.

8. A method for producing a protein, which comprises the steps of:
(a) culturing the host cell of claim 7 under the expression conditions;
(b) isolating the protein having the amino acid sequence of SEQ ID NO: 2 from the culture.

9. An antibody specifically bound with the protein of claim 1.

10. A pharmaceutical composition comprising a safe and efficient amount of the protein of claim 1 and a pharmaceutically acceptable carrier.


SEQUENCE LISTING (4 sequences)

SEQ ID NO: 1 - DNA, 1356 bp, Homo sapiens
atgcacggttactttggctgcaatgctgctgcagagcccggttactctgccttcgtgggaactccacagatatgtgtcacaaagatgtctacacggaactgccagggaatggactcagtgatcaaacccctggacacaattcctgaggataaaaaagtcagagttcagaggacacagagcacttttgacccatttgagaaaccagctaatcaagtaaagagggtgcattctgagaacaatgcttgcattaactttaagacctcctccactggcaaagagtcacctaaagttaggcggcactccagccccagctcgccaacaagtcccaaatttggaaaagctgactcatatgaaaagctggaaaaactaggggaaggatcttatgctacagtatacaaagggaaaagcaaggtaaatgggaagttggtagctctgaaggtgatcaggctgcaggaagaagaagggacacctttcacagctatcagggaagcttctcttttaaaaggactaaaacatgctaacatagtgctacttcatgacatcatccataccaaggagacgctgacacttgtgtttgaatatgtgcacactgatttatgtcagtacatggacaagcaccctggggggctgcatccagataatgtgaagttgtttttatttcagttgctgcgaggtctgtcttacatccaccagcgttatattttgcacagagacctgaaaccacagaaccttctgatcagtgacacgggggagttaaagctggcagatttcggtcttgcaagagcaaaatccgtccctagccacacatactccaacgaagtggttaccttgtggtacagacctccagatgtccttctaggctcaacagaatattccacctgccttgacatgtggggagtaggttgcatctttgttgaaatgatccaaggagttgctgcttttccaggaatgaaagacattcaggatcaacttgaacgaatatttctggttcttggaacaccaaatgaggacacatggcctggagttcattctttaccacattttaagccagaacgctttaccctgtacagctctaaaaaccttagacaagcatggaataagctcagctatgtgaaccatgcagaggacctggcctccaagctcctacaatgttccccaaagaacagactgtcggcacaggctgccttgagccacgagtattttagtgacctgccgccacggctatgggaactcaccgacatgtcttctatttttactgtcccaaatgtgagattgcaaccagaagctggagaaagcatgcgggcctttgggaaaaacaatagttatggcaaaagtctatcaaacagcaagcactga

SEQ ID NO: 2 - protein, 451 aa, Homo sapiens
MHGYFGCNAAAEPGYSAFVGTPQICVTKFSTRNCQGMDSVIKPLDTIPEDKKVRVQRTQSTFDPFEKPANQVKRVHSENNACINFKTSSTGKESPKVRRHSSPSSPTSPKFGKADSYEKLEKLGEGSYAYVYKGKSKVNGKLVALKVIRLQEEEGTPFTAIREASIIKGLKHANIVLLHDIIHTKETLTLVFEYYHTDLCQYMDKHPGGLHPDNVKLFLFQLLRGLSYIHQRYILHRDLKPGNLLISDTGELKLADFGLAKAKSVPSHTTSNEVVYLWYRPPDVLLGSTEYSTCLDMWGVGCIFVEMIQGVAAFPGMKDIQDQLERIFLVLGTPNEDTWPGVHSLPHFKPERFTLYSSKNLRQAWNKLSYVNHAEDLASKLLQCSPKNRLSAQAALSHEYFSDLPPRLWELTDMSSIFTVPNVRLQPEAGESMRAFGKNNSYGKSLSNSKH

SEQ ID NO: 3 - DNA, 39 bp, Artificial
  oligonucleotide
ggccaatccggccatgcacggttactttggctgcaatgc

SEQ ID NO: 4 - DNA, 43 bp, Artificial
  oligonucleotide
ggcctctaaggcctcagtgcttgctgtttgatagacttttgcc